(19) United States
(12) Patent Application Publication
(10) Pub. No.: US 2006/0067979 A1
(43) Pub. Date: Mar. 30, 2006

(54) Ophthalmic drug release device for multiple drug release

(75) Inventors: Jay F. Kunzler, Canandaigua, NY (US); Ronald J. Koch, Webster, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/236,947

(22) Filed: Sep. 28, 2005

Related U.S. Application Data
(60) Provisional application No. 60/614,615, filed on Sep. 30, 2004.

(51) Int. Cl. A61F 2/00

(52) U.S. Cl. 424/427

(57) ABSTRACT

A drug delivery device for placement in the eye includes a drug core comprising a pharmaceutically active agent, and a holder that holds the drug core. The holder is made of a material impermeable to passage of the active agent and includes an opening for passage of the pharmaceutically agent therethrough to eye tissue. The holder further comprises drugs of high water solubility.

### CROSS REFERENCE

This application claims the benefit of Provisional Patent Application No. 60/614,615 filed Sep. 30, 2004 and is incorporated herein by reference.

### FIELD OF THE INVENTION

This invention relates to a drug delivery device, preferably a device that is placed or implanted in the eye to release at least two pharmaceutically active agents of varying water solubility to the eye. The device includes a drug core and a holder for the drug core, wherein the holder is made of a material impermeable to passage of the active agent and includes at least one opening for passage of the pharmaceutical agent there through to the eye tissue. The holder also incorporates a drug. Both the central reservoir and the holder will deliver drugs of widely varying water (vitreous) solubility at a therapeutic level.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye for treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,375,972 (Guo et al.); U.S. Pat. No. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core having a pharmaceutically active agent and some type of holder for the drug core made of an impermeable material such as silicone or other hydrophobic materials. The holder includes one or more openings for passage of the pharmaceutically active agent through the impermeable material to eye tissue. Many of these devices include at least one layer of material permeable to the active agent, such as polyvinyl alcohol (PVA).

Previous drug delivery devices were only capable of delivering a drug or drugs that were incorporated into the drug core. This limited the devices primarily to delivery of drugs having relatively similar solubilities or in the case of varying solubilities; one drug would be delivered at a different rate or length of time than the other.

The advantage of this invention is that both the drug core and the holder for the core can be used for the delivery of drugs of widely varying water (vitreous) solubility. The drug holder is an unexpectedly ideal polymer for the delivery of drugs such as proteins, peptides, etc., of high water solubility

### BRIEF DESCRIPTION OF THE DRAWINGS

FIG. 1 is a side view of a first embodiment of a drug delivery device of this invention.

FIGS. 2-3 are graphical representations of the release rate of Timolol maleate from sample implants prepared according to the examples.

### SUMMARY OF THE INVENTION

According to a first embodiment, this invention relates to a drug delivery device for placement in the eye, comprising: a drug core comprising a pharmaceutically active agent; and a holder that holds the drug core, the holder being made of a material impermeable to passage of the active agent and including an opening for passage of the pharmaceutically active agent there through to eye tissue. Wherein the holder can deliver drugs of high water solubility, i.e., hydrophilic drug and wherein incorporation of the highly water soluble drugs into the drug core would result in rapid-release of the water soluble drug from the core. Incorporating the highly water soluble drug into the matrix of the drug holder has been to shown to allow for release of the drug of the drug at therapeutic levels for a desired period of time.

### DETAILED DESCRIPTION OF PREFERRED EMBODIMENTS

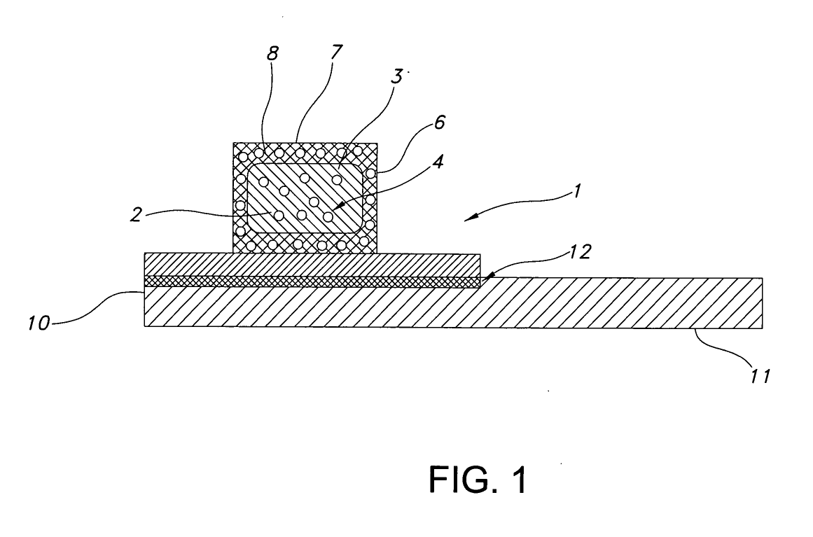

FIG. 1 illustrates a first embodiment of a device of this invention. Device 1 is a sustained release drug delivery device for implanting in the eye. Device 1 includes inner drug core 2 including a pharmaceutically active agent 3.

The active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility agents impeding such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal anti-inflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and antineovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; antiinflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eseridine salicylate, carbachol, diisopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

As shown in FIG. 1, active agent 3 may be mixed with a matrix material 4. Preferably, matrix material 4 is a polymeric material that is compatible with body fluids and the eye. Additionally, matrix material 4 should be permeable to passage of the active agent 3 therethrough, particularly when the device 1 is exposed to body fluids. For this embodiment, the matrix material 4 is PVA. Also, in other embodiments (not shown), inner drug core 2 may be coated with a coating of additional matrix material which may be the same or different from material 4 mixed with the active agent.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent 3 passes through any permeable matrix material 4 and permeable coating, and exits the device through passageway 7. For the illustrated embodiment, the holder 6 is made of silicone, especially polydimethylsiloxane (PDMS) material.

Holder 6 contains a hydrophilic drug 8 that allows for a different release profile than the active agent 3 in matrix material 4. Hydrophilic drugs would be those that have a strong tendency to bind or absorb water. Such drugs would include proteins, peptides, etc., Further examples of hydrophilic drugs would be those such as are listed in recognized treatises such as Ophthalmic Drug Facts and the PDR for Ophthalmic Medicines, the contents of both of which are incorporated by reference herein, that are capable of delivery by aqueous solution or gel.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

In addition to the illustrated materials, a wide variety of materials may be used to construct the devices of the present invention. The only requirements are that they are inert; non-immunogenic and of the desired permeability. Materials that may be suitable for fabricating the device 1 include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device 1 to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

Device 1 has a suture tab 10 having a suture hole 11 at one end thereof. The tab may be a monolithic aspect of device 1 or it may be adhered to the holder by adhesive 12.

According to certain embodiments, the holder is extracted to remove residual materials therefrom. For example, in the case of silicone, the holder may include lower molecular weight materials such as unreacted monomeric material and oligomers. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wettability of the holder and improve adherence of the drug core to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like or appropriate mixtures thereof including inert gases such as argon. Examples of mixed media include oxygen/argon or hydrogen/methanol. Typically, the plasma treatment is conducted in a closed chamber at an electric discharge frequency of 13.56 MHz, preferably between about 20 to 500 watts at a pressure of about 0.1 to 1.0 torr, preferably for about 10 seconds to about 10 minutes or more, more preferably about 1 to 10 minutes.

A device of the type shown in FIG. 1 may be manufactured as follows. The active agent may be provided in the form of a micronized powder, and then mixed with an aqueous solution of the matrix material, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

A cylindrical cup of silicone with unitary suture tab 10 is separately formed, for example by molding, having a size generally corresponding to the tablet and a shape as generally shown in FIG. 1. For example, the drug Timolol Maleate was polymerized at a 10% load with a Nusil® silicone resin (Med 6812) (obtained from NuSil Technologies, LLC, Carpinteria, Calif.). This formulation was shown to release the Timolol Maleate at a therapeutic level for up to one month. This Nusil® resin is well suited for molding (direct casting with drug) into silicone tubes that will provide for a transparent fit into currently used manufacturing procedures. When desirable, this silicone holder is then extracted with a solvent such as isopropanol. An opening 7 is placed in the silicone holder, for example, with a laser. If desired, a drop of liquid PVA may be placed into the holder through the opening 7 in the holder. Then, the inner drug core tablet is placed into the silicone holder through the same opening 7 and pressed into the cylindrical holder. If the drop of liquid PVA has been applied, the pressing of the tablet causes the liquid PVA to fill the space between the tablet inner core and the silicone holder, thus forming a permeable polymer cap (not shown).

It will be appreciated that the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

### EXAMPLE

Implant Preparation

Formulation

To Med 6-6812 Part A (1.0164 g.) (obtained from NuSil Technologies, LLC., Carpinteria, Calif.) and Med 6-6812 Part B (0.1052 g.) (obtained from NuSil Technologies, LLC., Carpinteria, Calif.) is added Timolol maleate (0.1014 g.) (commercially available) with mixing.

The mixture formed was injected into 0.022″ ID FEP fluoropolymer tubing with a syringe with a 23 gauge needle. The mixture in the tubing was cured at 65° C. for 15 hours.

Implants for study were pulled from the tubing after cure and cut into approximately 7 mm. lengths.

The implant formed comprised 8.30% Timolol Maleate (6.07% Timolol).

Testing

Initial release testing was conducted by placing 2 implants prepared according to the formulation above into three separate vials along with 3 mls of PBS (Phosphate buffered saline).

A (2.59 mg=214.7 mg Timolol Maleate=157.1 mg Timolol

B (2.59 mg=214.7 mg Timolol Maleate=157.1 mg Timolol

C (2.38 mg=197.5 mg Timolol Maleate=144.5 mg Timolol

The vials were then placed on a Titer Plate Shaker at 37° C. The PBS was exchanged at various time intervals and submitted for HPLC analysis to determine the Timolol concentration.

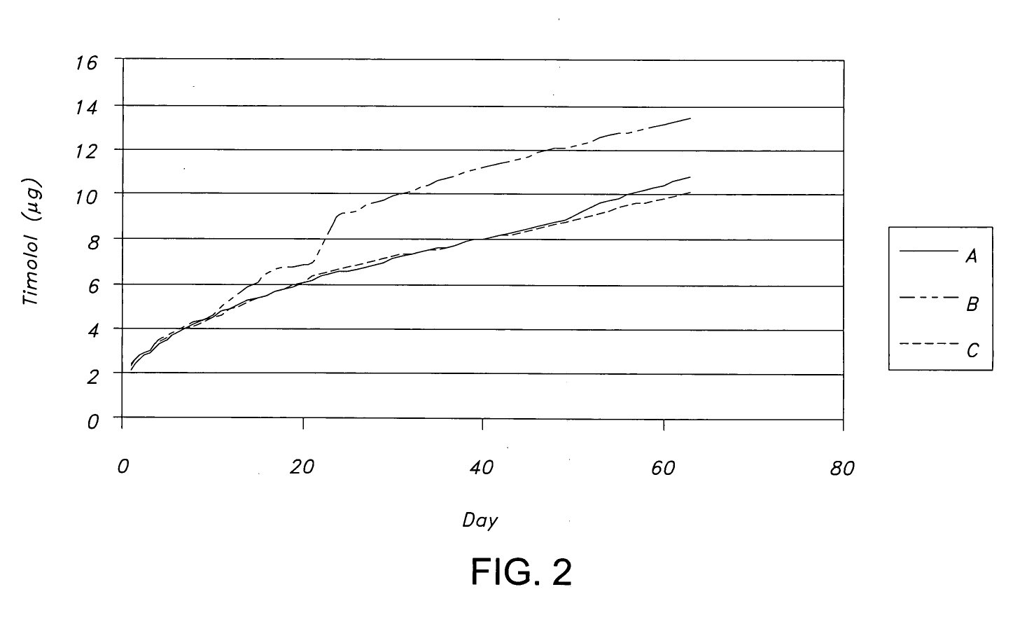

Results of this testing are provided in FIG. 2.

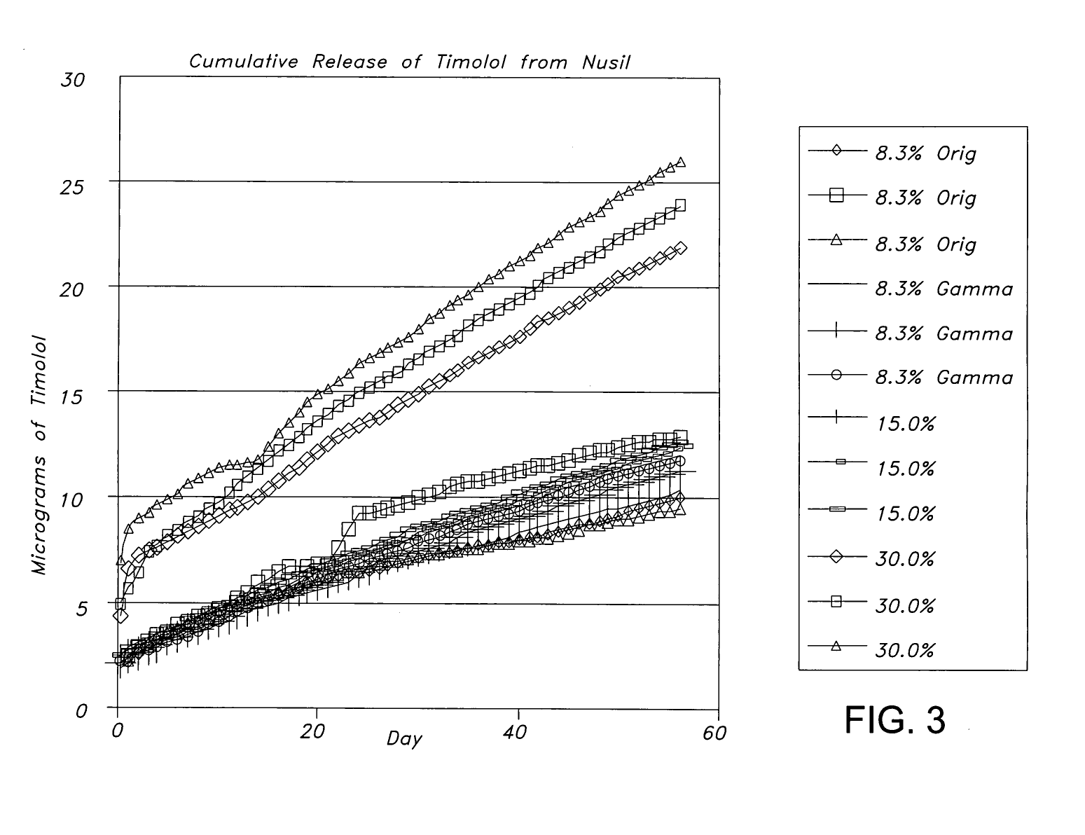

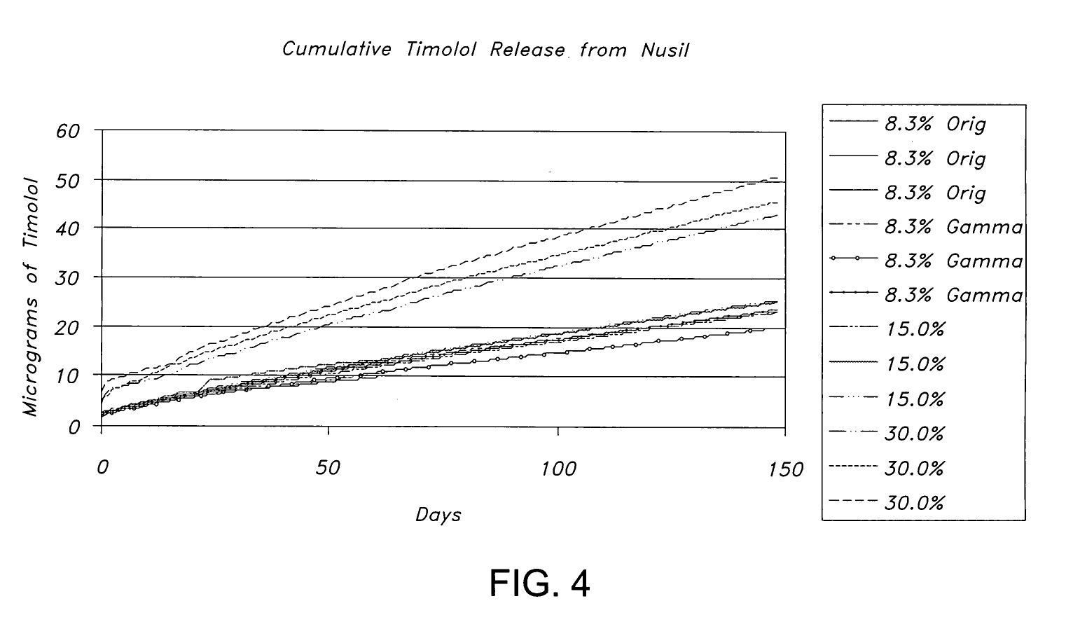

To test the effect of varying timolol maleate concentration on the release profile, implants were prepared as set forth above in the formulation section of the examples. Implants were prepared with varying concentrations of Timolol maleate to provide implants having concentrations of 8.3, 15 and 50 wt %. A portion of the 8.3 wt % Timolol maleate implants were subjected to standard gamma sterilization to determine if exposure to gamma irradiation would have an impact on the release profile. The results of this testing can be found in FIGS. 3 and 4.

Discussion

The results shown in FIGS. 3 and 4 demonstrate that gamma sterilization does not negatively affect the release profile of the implants prepared according to the formulation provided above. Although not wishing to be bound by a particular theory, the inventors believe that the reason that the 8.3 wt. % implants and the 15.0 wt. % implants have substantially the same release profile is because they were prepared on different days and therefore the Timolol maleate contained in the implants formed may not have had the same particle size. It is believed that the particle size of the Timolol maleate in the formulations is determined by the mixing conditions as the Timolol maleate used to make the formulations comprised a variety of particle sizes.

The examples and illustrated embodiment demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A drug delivery device for placement in the eye, comprising:
a drug core comprising a pharmaceutically active agent; and
a holder that holds the drug core, the holder being made of a material impermeable to passage of the active agent and having an opening therein and including a suture tab to aid in securing the device to the eye,
wherein the holder further comprises a drug of high water solubility.

2. The device of claim 1, wherein the impermeable material comprises silicone resin.

3. The device of claim 1, wherein the tab is adhered to at least one of the drug core and the holder.

4. The device of claim 1, wherein the tab is molded integrally with the holder.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder comprises a cylinder that surrounds the drug core and can deliver drugs of high water solubility in concert with the drugs of low water solubility contained in the drug core.

8. The device of claim 1, wherein the drug core is cylindrical.

9. The device of claim 1, wherein the drug core is coated with a material permeable to said active agent.

10. The device of claim 1, comprising a mixture of pharmaceutically active agents.
